(19) United States
(12) Patent Application Publication
(10) Pub. No.: US 2017/0204130 A1
(43) Pub. Date: Jul. 20, 2017

(54) FLAME RETARDANTS, PREPARATION METHODS, AND THERMOPLASTIC COMPOSITIONS THEREOF

(71) Applicant: E. I. DU PONT DE NEMOURS AND COMPANY, Wilmington, DE (US)

(72) Inventors: Xiao Pan, Shanghai (CN); Zhongning Zhang, Shanghai (CN); Gang Zuo, Shanghai (CN)

(21) Appl. No.: 15/314,724

(22) PCT Filed: May 30, 2014
(86) PCT No.: PCT/CN2014/078989

§ 371 Date: Nov. 29, 2016

(51) Int. Cl. C07H 15/26; C08K 5/5313; C09K 21/12

(52) U.S. Cl. CPC C07H 15/26; C09K 21/12; C08K 5/5313

(57) ABSTRACT

Disclosed are flame retardants comprising compounds of Formula 1, wherein the polyol is a disaccharide or a C₁₂ sugar alcohol, which has at least one glucose or one fructose unit per molecule, R¹ is H or CH₃; R² is H or CH₃; m is an integer ranging from 6 to 9; and n is an integer ranging from 2 to 9.

Also disclosed are methods for producing the inventive flame retardants, thermoplastic compositions and articles comprising the same, and methods for improving flame retardancy of thermoplastic polymers using the same.

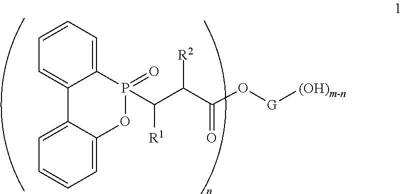

### FIELD OF THE INVENTION

The disclosure is related to novel class of sugar-based flame-retardants, compositions and articles comprising the same, and methods for decreasing the flammability of thermoplastic polymers using the same.

### BACKGROUND OF THE INVENTION

In the electronic industry, more and more metal parts are being replaced by polymeric parts due to their light weight and other favorable properties. However, one drawback that limits the even more wide use of polymeric parts is their inherent flammability. To solve this problem, various types of flame retardants have been developed for polymeric materials.

There are broad studies on adding known halogenated flame retardants to polymeric materials, however, halogenated flame retardants cause environmental pollution during manufacturing, recycling and disposing, and generate toxic and harmful gases during burning, therefore, halogenated flame retardants are gradually replaced by halogen-free flame retardants. Halogen-free flame retardants, especially phosphorus containing flame retardants have been widely used in polymeric materials, especially in polyesters and polyamides.

Phosphorus containing flame retardants include organic and inorganic materials covering a wide range of phosphorus compounds with different oxidation states, such as phosphates, phosphonates and phosphinates as well as red phosphorus. Among them, organic metal phosphinates (or organophosphorus metallic salt) are well suited for glass fiber reinforced polyamides and polyesters. For example, Exolit OP1230, diethylphosphinic acid aluminum disclosed in U.S. Pat. No. 6,534,673, can be applied in high temperature polyamides addressing balanced flame retarding performance and physical and electrical properties.

Polymer-based phosphorus containing flame retardants are known for enhancing compatibility with polymers and processability. There also have been efforts on synthesizing new polymers by incorporation of 9,10-dihydro-9-oxa-10-phosphor-phenanthrene-10-oxide (abbreviated as DOPO hereunder, CAS No. 35948-25-5) or its derivatives. For example, in U.S. Patent Publication No. US2010/0181696 discloses a DOPO containing polyester (Formula A) having a molecular weight, M. of more than 20,000 g/mol.

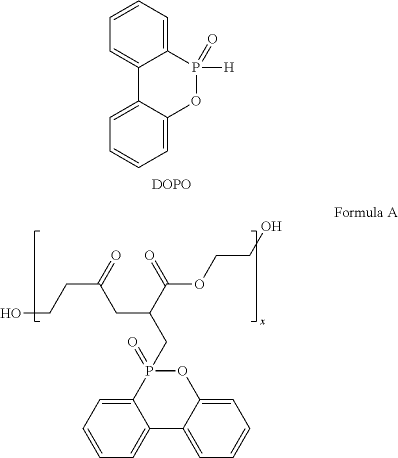

U.S. Pat. No. 8,236,881 also discloses many DOPO containing adducts including non-polymeric molecules. For example, DOPO adducts to acrylic esters of Formula B, wherein R′ represents the ester group of a polyhydroxy alcohol such as ethylene glycol, trimethylopropane, pentaerythritol or dipentaerythriol and y is a numeral from 2 to 6.

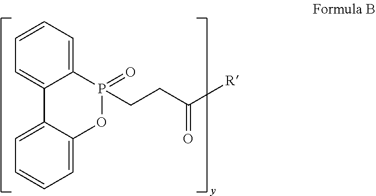

Currently, there still are needs for novel halogen-free flame retardants that have high flame retardancy, thermally stable for melt mixing with a wide range of thermoplastic polymers. Preferably, said novel halogen-free flame retardants are derived from renewably-sourced materials and may be manufactured by environmental friendly process.

### SUMMARY OF THE INVENTION

The present invention provides novel compounds of Formula 1, which can be used as flame retardants for polymeric materials:

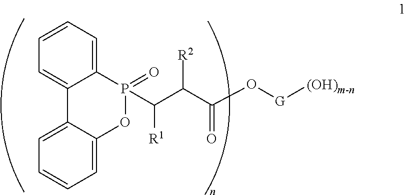

wherein
G-(OH)ₘ is a disaccharide or a C₁₂ sugar alcohol, which has at least one glucose or one fructose unit per molecule;
R¹ is H or CH₃;
R² is H or CH₃;
m is an integer ranging from 6 to 9; and
n is an integer ranging from 2 to 9.

The present invention also provides a method of preparing the compound of Formula 1, comprising:
i) forming a reaction mixture comprising an organophosphorus compound of Formula 2, a polyol of Formula 3, and a base,

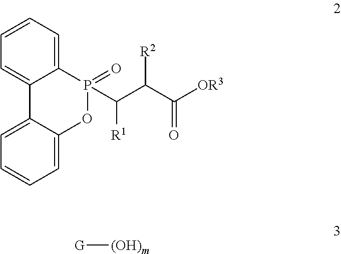

wherein
   R¹ is H or CH₃;
   R² is H or CH₃; and
   R³ is H or alkyl;
   the polyol is a disaccharide or a C12 sugar alcohol, which has at least one glucose or one fructose unit per molecule; and
   m is an integer ranging from 6 to 9;
ii) heating the reaction mixture at 125-230° C. and a pressure of 0.01-100 mbar for 8-24 hours; and
iii) isolating a mixture of the compound of Formula 1.

The present invention further provides a thermoplastic composition having improved flame retardancy, comprising:
a) 80 to 99.9% by weight of a thermoplastic polymer, and
b) 0.1 to 20% by weight of the compound of Formula 1,
wherein the % is based on the total weight of the thermoplastic composition.

Provided herein also relates to an article comprising or produced from the thermoplastic compositions mentioned above.

Furthermore, this invention provides a method for improving flame retardancy of a thermoplastic polymer comprising: incorporating a flame retardant composed of the compounds of Formula 1 into the thermoplastic polymer.

### DETAILS OF THE INVENTION

All publications, patent applications, patents and other references mentioned herein, if not otherwise indicated, are explicitly incorporated by reference herein in their entirety for all purposes as if fully set forth.

Unless otherwise defined, all technical and scientific terms used herein have the same meaning as commonly understood by one of ordinary skill in the art to which this invention belongs. In case of conflict, the present specification, including definitions, will control.

Unless stated otherwise, all percentages, parts, ratios, etc., are by weight.
“mol %” refers to mole percent.

When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

As used herein, the term “produced from” is synonymous to “comprising”. As used herein, the terms “includes”, “including”, “comprises”, “comprising”, “has”, “having”, “contains” or “containing”, or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The materials, methods, and examples herein are illustrative only and, except as specifically stated, are not intended to be limiting. Although methods and materials similar or equivalent to those described herein can be used in the practice or testing of the present invention, suitable methods and materials are described herein.

When the term “about” is used in describing a value or an end-point of a range, the disclosure should be understood to include the specific value or end-point referred to.

The flame retardant compounds of the present invention and methods for producing the same are described in detail hereinunder. Also disclosed are thermoplastic compositions and articles comprising the same, and methods for improving flame retardancy of polymeric materials using the same.

The flame retardant compounds of Formula 1 disclosed herein are generally prepared by the following methods and variations as described in Scheme 1.

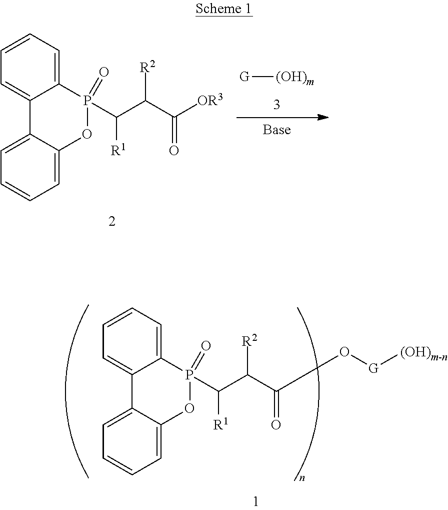

As shown in Scheme 1, a method for preparing a compounds of Formula 1 comprises the step of i) forming a reaction mixture comprising an organophosphorus compound of Formula 2, a polyol of Formula 3, and a base, and ii) heating the reaction mixture. The definitions of R¹, R², R³, m and n for the compounds of Formula 1 and the organophosphorus compound of Formula 2 are as defined above in the Summary of the Invention unless indicated otherwise.

The polyol of Formula 3 as represented by the general structure: G-(OH)ₘ is a disaccharide or a C₁₂ sugar alcohol, which has at least one glucose or one fructose unit per molecule, and m is an integer of from 6 to 9. The polyol of Formula 3 may react with the organophosphorus compound of Formula 2 through direct esterification (i.e. when R³ is H) or transesterification (i.e. when R³ is C₁-C₄ alkyl).

The molar ratio of the organophosphorus compound of Formula 2 to the hydroxyl groups of the polyol of Formula 3 is preferably 0.5:1 to 1.5:1. For example, when the polyol of Formula 3 is sucrose having 8 hydroxyl groups per molecule, then the mole ratio of organophosphorus compound of Formula 2 to sucrose is at least about 4:1, more preferably 8:1, and most preferably about 12:1. For another example, when the polyol of Formula 3 is isomalt having 9 hydroxyl groups per molecule, then the mole ratio of organophosphorus compound of Formula 2 to isomalt is at least about 4.5:1, preferably 9:1, and more preferably about 13.5:1.

For the direct esterification processes of the invention, suitable base to be used as a reaction catalyst include alkali metals such as sodium, lithium and potassium; alloys of two or more alkali metals such as sodium-lithium and sodium-potassium alloys; alkali metal hydrides, such as sodium, lithium and potassium hydride; alkali metal lower (C₁-C₄) alkyls such as butyl lithium; and alkali metal alkoxides of lower (C₁-C₄) alcohols, such as lithium methoxide, potassium t-butoxide, potassium methoxide, and/or sodium methoxide. Suitable base to be used as a reaction catalyst in the transesterification processes of the invention include carbonates and bicarbonates of alkali metals or alkaline earth metals, for example, sodium carbonate, potassium carbonate, calcium carbonate, barium carbonate, and sodium bicarbonate. In one embodiment of the invention, the base includes potassium carbonate, sodium carbonate, barium carbonate, or mixtures of these compounds having particle sizes that are less than about 100 microns, preferably less than about 50 microns.

As the transesterification process may be catalyzed by a weaker base and run under a milder condition as compared to those of the direct esterification, the transesterification is the preferred method for preparing the compounds of Formula 1.

For the transesterification reaction, the amount of the base is typically from about 0.01 c to about 0.5 moles per mole of the polyol of Formula 3. In one embodiment, the molar ratio of base to polyol is from about 0.01:1 to about 0.1:1; preferably from about 0.02:1 to about 0.05:1; more preferably, from about 0.1:1 to about 0.3:1.

In one embodiment of the inventive process, a mixture of a polyol of Formula 3, a base selected from potassium carbonate, sodium carbonate, barium carbonate and mixtures thereof, and excess organophosphorus compound of Formula 2 is heated to form the compound of Formula 1.

In another embodiment the transesterification reaction occurs in one step. The entire desired amount of the organophosphorus compound of Formula 2 is mixed with polyol of Formula 3, and base to form a reaction mixture and the reaction mixture is then heated. There is no additional organophosphorus compound of Formula 2 added at a later reaction point.

The reaction mixture may be substantially free of added solvent, or may be free of added solvent. As used herein “added solvent” refers to solvent added to the reaction mixture, and is not intended to include any alcohol formed as the by-product during the transesterification. The term “substantially free of added solvent” is intended to refer to reaction mixtures having no more than 1% by weight of the mixture to be the added solvent.

Sometimes, solvent may be added to the reaction mixture to facilitate the reaction progress. Suitable solvents include dimethylformamide (DMF), formamide, dimethyl sulfoxide, and pyridine. Dimethyl sulfoxide is one of the preferred solvent.

While the added solvent has to be removed later from the products, it's best to keep the amount of the solvent to the minimum. In one embodiment, the amount of the solvent in no more than 10% by weight, or 7.5% by weight, or 5% by weight of the combined weight of the reactants.

The reaction mixture is heated to a temperature sufficient to allow reaction between the polyol of Formula 3 and the organophosphorus compound of Formula 2 and to complete said reaction in an efficient manner. As the transesterification reaction proceeds, a C₁-C₄ alkyl alcohol (e.g., methanol or ethanol) is formed as a by-product. In order to promote the reaction, the alcohol by-product is preferably removed from the reaction mixture. Without being limited by theory, it is believed that reducing the partial pressure of the lower alcohol in the headspace below what is in equilibrium with the liquid phase will result in alcohol removal from the liquid phase reaction mixture. Therefore, it is generally advantageous to reflux the reaction mixture (i.e., separate the alcohol by-product from the vapor phase leaving the reaction and return them to the reaction mixture). Refluxing may be performed using a mechanical refluxing system such as, for example, a reflux column, or by distilling off the alcohol by-product and returning the organophosphorus compound of Formula 2 to the reaction mixture.

In one embodiment, the reaction mixture is heated to a temperature of at least about 125° C. In another embodiment, the reaction mixture is heated to a temperature in the range of from about 125° C. to about 230° C., or from about 150° C. to about 210° C., or from about 170° C. to about 190° C.

The reaction mixture is heated under a pressure sufficient to facilitate the reaction and, as noted above, may be below, at or above atmospheric pressure. In one embodiment, the pressure is sufficient to reflux excess organophosphorus compound of Formula 2 during the reaction as disclosed above. In another embodiment, the reaction mixture is heated under a pressure sufficient to maintain a substantially constant reflux rate of the organophosphorus compound of Formula 2.

In one embodiment, the esterification or transesterification is conducted at a pressure of from about 0.01 mbar to about 100 mbar without inert gas sparge, preferably from about 0.1 mbar to about 10 mbar. In another embodiment, the esterification or transesterification is conducted at a pressure of from about 0.01 to about 500 mbar utilizing a nitrogen sparge to keep the combined partial pressures of the organophosphorus compound of Formula 2 and lower alcohol in a range of from about 0.01 to about 100 mbar. In a further embodiment, the esterification or transesterification is conducted at a pressure of from about 0.1 to about 400 mbar utilizing a nitrogen sparge to keep the combined partial pressures of lower alkyl ester and lower alcohol in a range of from about 0.1 to about 50 mbar.

Many techniques known in the art can be used effectively and efficiently to reduce the partial pressure of the lower alcohol. Vacuum, with or without inert gas sparging into the vapor or liquid phases, can be used to remove the alcohol and promote the reaction. Alternatively, inert gas sparging can be used at atmospheric or greater pressures to promote alcohol removal. Sparging inert gas into the liquid has the added benefit of increasing surface area available for mass transfer of lower alcohol into the gas phase. As inert gas sparging is increased, the vacuum level may be decreased in order to achieve a desired lower alcohol partial pressure.

The transesterification reaction between the polyol of Formula 3 and the organophosphorus compound of Formula 2 can be conducted in any reactor conventionally employed, including, but not limited to batch, semi-batch and continuous reactors. Column reactors, packed or multi-stage, are suitable for use in the transesterification reaction. Plug flow column reactors are also suitable.

The esterification or transesterification typically run for around 2-24 hours. After the reaction completion, the resulting crude product comprising a mixture of compounds of Formula 1 that can be isolated and purified by known techniques e.g., precipitation, filtration, centrifugation, etc. to remove the base and unreacted/excess starting materials.

The organophosphorus compounds of Formula 2 used in this invention are known and may be prepared by the method disclosed in U.S. Pat. No. 4,280,951, as shown in Scheme 2.

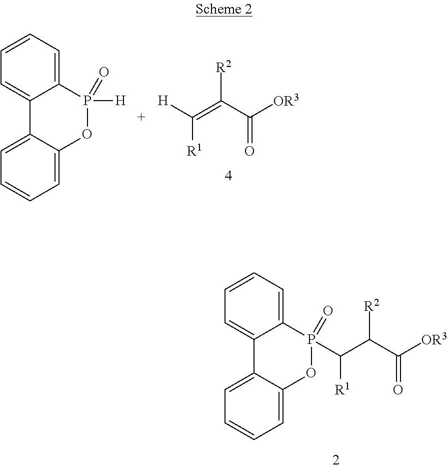

By adding an acrylate of Formula 4 to DOPO, the adduct, i.e. an organophosphorus compound of Formula 2 may be prepared. Examples of acrylates of Formula 4 include acrylic acid, methacrylic acid, crotonic acid and alkyl esters thereof, such as methyl acrylate, ethyl acrylate, butyl acrylate, octyl acrylate, methyl methacrylate, ethyl methacrylate, propyl methacrylate, butyl methacrylate, octyl methacrylate, methyl crotonate, ethyl crotonate, butyl crotonate and octyl crotonate. In one embodiment of the present invention, the acrylate of Formula 4 is acrylic acid, methacrylic acid, methyl acrylate, ethyl acrylate, methyl methacrylate, or ethyl methacrylate.

The adduct forming reaction generally proceeds quantitatively. Since polymerization of acrylic acid, methacrylic acid and esters thereof may take place simultaneously with the adduct forming reaction. To minimize the polymerization, it is preferred to add the acrylates of Formula 4 in a controlled rate to DOPO as the reaction advances. Further, the polymerization reaction may be prevented by adding a small amount of polymerization inhibitor to the reaction system.

The adduct forming reaction may proceed with or without a catalyst at temperatures of from 110° C. to 180° C. for 2-10 hours. The unreacted acrylates of Formula 4 is removed from the reaction products under vacuum or, if desired, the reaction products are purified by a solvent recrystallization method.

Alternatively, the organophosphorus compounds of Formula 2 may be purchased from commercial sources, for example, Eutec Chemical Co., LTD.

As demonstrated by the examples below, the compounds of Formula 1 disclosed herein, when incorporated into thermoplastic polymers (such as polyesters, polyamides), can improve the flame retardancy thereof. Therefore, further disclosed herein are flame-retardant thermoplastic compositions comprising at least one thermoplastic polymer and the compounds of Formula 1.

The flame retardant composition may comprise about 0.1 weight % to about 20 weight %; or about 1 weight % to about 18 weight %, or about 5-15 weight % of the compounds of Formula 1, wherein the weight % is based on the total weight of the flame retardant composition.

The thermoplastic polymers used herein may be any suitable thermoplastic polymers. In accordance with the present disclosure, suitable thermoplastic polymers used herein are selected from polyesters, polyester elastomers, polyamides, polyurethanes, polyolefins, and blends thereof.

According to the present invention, the polyester constitutes any condensation polymerization products derived from, by esterification or transesterification, a diol and a dicarboxylic acid including an ester thereof.

Examples of such diols include glycols having 2 to about 10 carbon atoms such as ethylene glycol, 1,2-propanediol, 1,3-propanediol, 1,3-butanediol, 1,4-butanediol, 1,5-pentanediol, 2,2-dimethyl-1,3-propanediol, 1,6-hexanediol, diethylene glycol, triethylene glycol, polyethylene glycol, 1,2-, 1,3- and 1,4-cyclohexane dimethanol, and longer chain diols and polyols, such as polytetramethylether glycol, which are the reaction products of diols or polyols with alkylene oxides, or combinations of two or more thereof.

Examples of such a dicarboxylic acids include terephthalic acid, isophthalic acid, phthalic acid, succinic acid, glutaric acid, adipic acid, azelaic acid, sebacic acid, 1,4-cyclohexane dicarboxylic acid, 1,3-cyclohexane dicarboxylic acid, 1,12-dodecanedioic acid, and the derivatives thereof such as the dimethyl-, diethyl-, dipropyl esters of these dicarboxylic acids, or combinations of two or more thereof.

The polyester may be a homopolymer or a copolymer. When the copolymer is adopted, the dicarboxylic acid component constituting the copolymer may be prepared from one or more compounds selected from: (1) linear, cyclic, and branched aliphatic dicarboxylic acids having 4 to 12 carbon atoms, such as succinic acid, glutaric acid, adipic acid, azelaic acid, sebacic acid, 1,12-dodecanedioic acid, 1,4-cyclohexane dicarboxylic acid; from (2) aromatic dicarboxylic acids having 8 to 12 carbon atoms, such as phthalic acid, isophthalic acid, terephthalic acid or 2,6-naphthalene dicarboxylic acid; or ester-forming equivalents of these. In addition, the diol component constituting the copolymer may be prepared from one or more compounds selected from: (3) linear, cyclic, and branched aliphatic diols having 2 to 8 carbon atoms, such as ethylene glycol, 1,2-propanediol, 1,3-propanediol, 1,4-butanediol, 1,6-hexanediol, 3-methyl-1,5-pentanediol, 2,2-dimethyl-1,3-propanediol, 2-methyl-1,3-propanediol, 1,4-cyclohexane dimethanol or 1,4-cyclohexanediol; and from: (4) aliphatic and aromatic ether glycols having 4 to 10 carbon atoms, such as hydroquinone bis(2-hydroxyethyl)ether.

These dicarboxylic acids and/or diols may be used either singly or in the form of a mixture of two or more copolymerized units. The major copolymerized unit may be present in the copolymer at least about 60 mol %; preferably, about 70 mol % or more.

In one embodiment, the thermoplastic polymers used in the inventive thermoplastic composition are polyester homopolymers or polyester copolymers having two or more copolymerized units, where the amount of the major copolymerized unit is at least about 70 mol % in the copolymer. In another embodiment, the thermoplastic polymers used in the inventive thermoplastic composition are polyesters including polyethylene terephthalate (PET), polytrimethylene terephthalate (PTT), polybutylene terephthalate (PBT), polyethylene naphthalate (PEN), polybutylene naphthalate (PBN), and polycyclohexylenedimethylene terephthalate (PCT)). In yet another embodiment, suitable thermoplastic polymers are polyesters including PET, PTT, PBT, and blends thereof. In a further embodiment, suitable thermoplastic polymers are polyester elastomers including copolyetherester.

The above mentioned polyesters used herein may also be obtained commercially from various vendors. For example, suitable PET may be obtained commercially from E.I. du Pont de Nemours and Company (U.S.A.) (hereafter “DuPont”) under the trade name Rynite®, from Far Eastern Industry (Shanghai) Ltd. under the trade name Eastlon®; suitable PTT may be obtained commercially from DuPont under the trade name Sorona®; suitable PBT may be obtained commercially from DuPont under the trade name Crastin®, from BASF under the trade name Ultradur®, from Chang Chun Plastics Co. Ltd. under the trade name Longlite®; suitable PCT may be obtained commercially from Ticona (The Netherland) under the trade name Thermx™; and suitable copolyetheresters may be obtained commercially from DuPont under the trade name Hytrel®.

In accordance with the present disclosure, suitable polyamides include both aliphatic polyamides and semi-aromatic polyamides.

Polyamides are (a) condensation products of one or more dicarboxylic acids and one or more diamines, or (b) condensation products of one or more aminocarboxylic acids, or (c) ring opening polymerization products of one or more cyclic lactams. The semi-aromatic polyamides used herein may be homopolymers, copolymers, terpolymers or higher polymers containing at least one aromatic monomer component. For example, a semi-aromatic polyamide may be obtained by using an aliphatic dicarboxylic acid and an aromatic diamine, or an aromatic dicarboxylic acid and an aliphatic diamine as starting materials and subjecting them to polycondensation.

Suitable diamines used herein may be selected from aliphatic diamines, alicyclic diamines, and aromatic diamines. Exemplary diamines useful herein include, without limitation, tetramethylenediamine; hexamethylenediamine; 2-methylpentamethylenediamine; nonamethylenediamine; undecamethylenediamine; dodecamethylenediamine; 2,2,4-trimethylhexamethylenediamine; 2,4,4-trimethylhexamethylenediamine; 5-methylnona-methylenedi amine; 1,3-bis(aminomethyl)cyclohexane; 1,4-bis(aminomethyl)-cyclohexane; 1-amino-3-aminomethyl-3,5,5-trimethylcyclohexane; bis(4-aminocyclohexyl)-methane; bis(3-methyl-4-aminocyclohexyl)methane; 2,2-bis(4-aminocyclohexyl)propane; bis(amino-propyl)piperazine; aminoethylpiperazine; bis(p-aminocyclohexyl)methane; 2-methyl-octamethylenediamine; trimethylhexamethylenediamine; 1,8-diaminooctane; 1,9-diamino-nonane; 1,10-diaminodecane; 1,12-diaminododecane; m-xylylenediamine; p-xylylenediamine; and the like and derivatives thereof.

Suitable dicarboxylic acids used herein may be selected from aliphatic dicarboxylic acids, alicyclic dicarboxylic acids, and aromatic dicarboxylic acids. Exemplary dicarboxylic acids useful herein include, without limitation, adipic acid; sebacic acid; azelaic acid; dodecanedoic acid; terephthalic acid; isophthalic acid; phthalic acid; glutaric acid; pimelic acid; suberic acid; 1,4-cyclohexanedicarboxylic acid; naphthalenedicarboxylic acid; and the like and the like and derivatives thereof.

Exemplary aliphatic polyamides used herein include, without limitation, polyamide 6; polyamide 6,6; polyamide 4,6; polyamide 6,10; polyamide 6,12; polyamide 11; polyamide 12; polyamide 9,10; polyamide 9,12; polyamide 9,13; polyamide 9,14; polyamide 9,15; polyamide 6,16; polyamide 9,36; polyamide 10,10; polyamide 10,12; polyamide 10,13; polyamide 10,14; polyamide 12,10; polyamide 12,12; polyamide 12,13; polyamide 12,14; polyamide 6,14; polyamide 6,13; polyamide 6,15; polyamide 6,16; polyamide 6,13; poly(dimethyldiaminodicyclohexylmethane dodecanamide) (polyamide MACM, 12); and the like.

Exemplary semi-aromatic polyamides used herein include, without limitation, poly(m-xylylene adipamide) (polyamide MXD,6); poly(m-xylylene terephthalamide) (polyamide MXD,T); poly(m-xylylene isophthalamide) (polyamide MXD,I); poly(2-methylpentamethylene terephthalamide) (polyamide D,T); poly(dimethyldiaminodicyclohexylmethane terephthalamide) (polyamide MACM,T); poly(dimethyldiaminodicyclohexylmethane isophthalamide) (polyamide MACM,I); poly(dodecamethylene terephthalamide) (polyamide 12,T); poly(dodecamethylene isophthalamide) (polyamide 12,1); poly(undecamethylene terephthalamide) (polyamide 11,T); poly(decamethylene terephthalamide) (polyamide 10,T); poly(nonamethylene terephthalamide) (polyamide 9,T); poly(hexamethylene terephthalamide) (polyamide 6,T); and poly(hexamethylene isophthalamide) (polyamide 6,1).

Exemplary copolyamides used herein include, without limitation, polyamide 6,T/6,6 (i.e., having at least about 50 mol % of its repeating units derived from 6,T); polyamide 6,6/6,T (i.e., having at least about 50 mol % of its repeating units derived from 6,6); polyamide 6,T/6,I (i.e., having at least about 50 mol % of its repeating units derived from 6,T); polyamide 6,I/6,T, (i.e., having at least about 50 mol % of its repeating units derived from 6,1); polyamide 6,T/D,T; polyamide 6/6,T; polyamide 6,6/6,T/6,I; polyamide MXD,I/6,I; polyamide MXD,I/12,I; polyamide MXD,I/MXD,T/6,I/6,T; polyamide MACM,I/12; polyamide MACM,I/MACM,12; polyamide MACM,I/MACM,T/12; polyamide 6,I/MACM,I/12; polyamide 6,I/6,T/MACM,I/MACM,T; polyamide 6,I/6,T/MACM,I/MACM,T/12; and the like.

In the flame retardant composition, the thermoplastic polymer may be present at a level of about 80 weight % to about 99.9 weight %, or about 82 weight % to about 99 weight %, or about 85 weight % to 95 weight %, wherein the weight % is based on the total weight of the flame retardant composition.

In one embodiment, the flame retardant compositions disclosed herein may further comprise one or more additional flame retardants. The one or more additional flame retardants that may be used in combination with the compounds of Formula 1 and may be selected from any suitable flame retardants known in the art. For example, the additional flame retardants used herein may include, without limitation,
halogen-containing flame-retardants, such as, tetrabromobisphenol A (TBBA), tetrabromo phthalic anhydride (TBPA), tetrabromobisphenol A bis(2,3-dibromopropyl ether) (BDDP), hexabromocyclododecane (HBCD), decabromodiphenyl ether (DBDE), 1,2-bis(pentabromophenyl) ethane (DBDPE), tris(2,3-dibromopropyl)isocyanurate (TBC), dodecachloropentacyclo-octadecadiene (Dechlorane plus), chlorinated paraffins, etc.;
inorganic flame retardants, such as, magnesium hydroxide, aluminum hydroxide, antimony oxide, zinc borate, etc.;
phosphorus-containing flame-retardants (such as red phosphorus, resorcinol bis(diphenyl phosphate) (RDP), bisphenol A bis(diphenyl phosphate) (BDP), resorcinol bis(2,6-dixylenyl phosphate) (RDX), triphenyl phosphate (TPP), tributyl phosphate (TBP), (1-oxo-4-hydroxymethyl-2,6,7-trioxa-l-phospho-bicyclo[2.2.2]octane (PEPA), dimethyl methyl phosphonate (DMMP), 9,10-dihydro-9-oxa-10-phosphaphenanthrene-10-oxide (DOPO), aluminum diethylphosphinate, zinc diethylphosphinate, ammonium polyphosphate (APP), etc.; and
nitrogen-containing compounds, such as, melamine polyphosphate (MPP), melamine (MA), melamine cyanurate (MC), etc.

Preferably, the additional flame retardants used in the present flame retardant compositions also are free of halogen.

The flame retardant thermoplastic compositions disclosed herein may further comprise other additives, such as colorants, antioxidants, UV stabilizers, UV absorbers, heat stabilizers, lubricants, tougheners, impact modifiers, reinforcing agents, viscosity modifiers, nucleating agents, plasticizers, mold release agents, scratch and mar modifiers, impact modifiers, emulsifiers, pigments, optical brighteners, antistatic agents, fillers, and combinations of two or more thereof.

Suitable fillers may be selected from calcium carbonates, silicates, talcum, carbon black, and combinations thereof.

Based on the total weight of the flame retardant composition disclosed herein, such additional additive(s) may be present at a level of about 0.01 weight % to about 20 weight %, or about 0.01 weight % to about 10 weight %, or about 0.2 weight % to about 5 weight %, or about 0.5 weight % to about 2 weight %, so long as they do not detract from the basic and novel characteristics of the flame retardant compositions and do not significantly adversely affect the performance of the flame retardant compositions.

The flame retardant thermoplastic composition disclosed herein may be prepared by any suitable process. For example, the compounds of Formula 1 may be introduced into a melt of the thermoplastic polymer (s) by a melt blending process. And the melt blending process may be carried out using any suitable blending (or compounding) device, such as a kneader, extruder, or a mixer. Preferably, the flame retardant thermoplastic composition disclosed herein are melt-mixed blends, wherein all of the polymeric components are well-dispersed within each other and all of the non-polymeric ingredients are homogeneously dispersed in and bound by the polymeric matrix, such that the blend forms a unified whole.

As demonstrated by the examples below, the compound of Formula 1 has high char yield and excellent thermal stability and can be used to improve flame retardant properties in thermoplastic polymers.

Yet further disclosed herein are articles comprising one or more component parts formed of the thermoplastic compositions disclosed herein, wherein the articles include, without limitation, motorized vehicles, electrical/electronic devices, furniture, footwear, roof structure, outdoor apparels, and water management system, etc.

### EXAMPLES

Without further elaboration, it is believed that one skilled in the art using the preceding description can utilize the present invention to its fullest extent. The following Examples are, therefore, to be construed as merely illustrative and not limiting of the disclosure in any way whatsoever. Steps in the following Synthetic Examples illustrate a general procedure for each step in an overall synthetic transformation, and the starting material for each step may not have necessarily been prepared by a particular preparative run whose procedure is described in other Examples or Steps.

Materials

The ingredients used in the synthetic examples, examples and comparative examples of the flame retardant thermoplastic compositions are given in Table 1.

TABLE 1

| Abbreviation | Material |
|---|---|
| PTT | SORONA ®, a polytrimethylene terephthalate homopolymer with a melting |
| | temperature (mp) of 228° C., and intrinsic viscosity (IV) of 1.02 dL/g, obtained from |
| | DuPont. |
| PBT | Longlite ® (1100 211H), a polybutylene terephthalate homopolymer with a mp of |
| | 225° C., and intrinsic viscosity (IV) of 1.1, purchased from Chang Chun Plastics Co. |
| | Ltd., Taiwan. |
| PET | Eastlon ® CB600, a polyethylene terephthalate homopolymer with a mp of 245° C., and |
| | intrinsic viscosity (IV) of 0.75, purchased from Far Eastern Industry (Shanghai) Ltd. |
| PA610 | a polyamide 6,10, with middle viscosity 0604 DX, obtained from DuPont. |
| PA66 | Zytel ®101 NC010, a polyamide 6,6, obtained from DuPont. |
| PA6 | Durethan ® B29 RV50, a polyamide 6, obtained from Lanxess. |
| Potassium carbonate | Anhydrous white powder (CAS number 5894-08-7), purchased from SCRC. |
| Compound 2a | 6H-Dibenz[c,e][1,2]oxaphosphorin-6-oxide-6-propionate methyl ester (CAS number |
| | 63562-42-5), purchased from Eutec Chemical Co., LTD |
| Compound 2b | 6H-Dibenz[c,e][1,2]oxaphosphorin-6-oxide-6-(α-methyl)propionate methyl ester |
| | (CAS number 144137-53-1), purchased from Eutec Chemical Co., LTD. |
| ME-P8 | A polymeric DOPO containing flame retardant (CAS number 403614-60-8), |
| | purchased from Eutec Chemical Co., LTD., the number average molecular weight |
| | was about 10,000, and the phosphorus content is 7.8-8.2%. |
| OP1230 | Diethylphosphinic acid aluminum (CAS number 225789-38-8), a type of |
| | organophosphorus salt in white powers, purchased from Clariant. |
| Isomalt | (2ξ)-6-O-α-D-Glucopyranosyl-D-arabino-hexitol (CAS number 64519-82-0), a C₁₂ |
| | sugar alcohol purchased from Darui Fine Chemical. |
| Sucrose | α-D-glucopyranosyl-β-D-fructofuranoside (CAS number 57-50-1), a disaccharide |
| | purchased from Sigma Aldrich. |
| Pentaerythritol | CAS number 115-77-5, purchased from SCRC. |
| IRGANOX ® 1010 | Tetrakis(methylene-3-(3,5-di-t-butyl-4-hydroxyphneyl)propionate)methane (CAS |
| | number 6683-19-8), a phenolic based antioxidant, purchased from BASF. |
| IRGANOX ® 1098 | A hindered phenolic antioxidant (CAS number 23128-74-7), purchased from BASF. |

### General Testing Methods

Phosphorus content: a testing solution was prepared by adding 8 mL of conc. HNO₃ (about 65%) to 0.1 g of a sample to digest it in a microwave digestion instrument for 30 minutes at 150° C. The resulting sample solution was diluted with an 2% HNO₃ aqueous solution to 2500 mL, and was added with a Scandium aqueous solution (1 mg/L) as internal standard. The analysis was performed on an inductively coupled plasma (ICP) system (PE Optima™ 7000 DV, manufactured by Perkin Elmer).

Glass transition temperature (Tg) was determined by differential scanning calorimetry (DSC) analysis, which was carried out with a TA Q100 differential scanning calorimeter in a dry nitrogen atmosphere. The instrument was equilibrated at 35° C., first heated to 150° C. at a heating rate of 10° C./min, and held at this temperature for 1 min, marked the end of heating cycle 1, followed by cooling down at a rate of 10° C./min to 35° C. and held at this temperature for 5 min. A heating rate of 10° C./min was used for the 2^{nd} heating cycle to 150° C., and held at this temperature for 1 min, and the T_{g} data were taken on this cycle for all exemplified synthetic samples.

Thermal decomposition temperature (T_{d}) was determined by thermal gravimetric analysis (TGA) analysis, which was carried out with a TA Q500 instrument at a heating rate of 20° C./min in a temperature range of 35-700° C. under air atmosphere. T_{d} is the temperature corresponding to the interception point of the extended initial experimental baseline and the tangent line of the maximum weight loss of the thermogravimetry curve.

Mechanical properties such as maximum tensile stress, tensile stress at break, and tensile strain at break were measured on universal material testing machine Instron 5567 according to ISO527:1993(E).

Flex properties was tested on universal material testing machine Instron 5567 according to ISO178:2001(E).

N-charpy impact was tested on CEAST impact tester according to ISO179.

### Synthetic Example 1. Preparation of Compound 1a

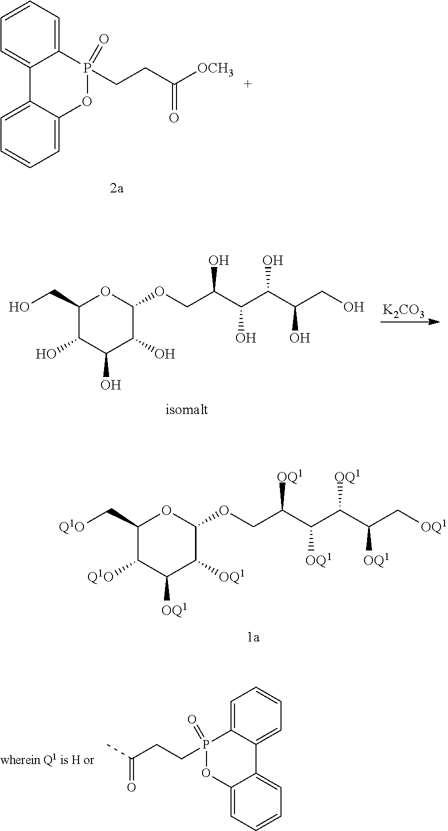

A mixture of 81.37 g (0.236 moles) of isomalt, 500 g (1.654 moles) of Compound 2a (pre-melted by warming in an 80° C. water bath), and 8.72 g (0.063 moles) of potassium carbonate was placed in a 1000 mL three-necked flask equipped with mechanical stirrer, a nitrogen inlet, a condenser and a byproduct collecting flask. The collecting flask was submerged in a dry ice/isopropyl alcohol bath and connected to a vacuum pump. The molar ratio of Compound 2a to isomalt was about 7:1.

The reaction mixture was heated to melt completely at about 170° C. under nitrogen protection with mechanical stirring. After heating at about 170° C. for 1 hour, the reaction temperature was increased to about 190° C. and the nitrogen flow was stopped. The methanol byproduct was removed from the reaction mixture under reduced pressure (about 10⁻¹-10⁻² mbar) and was collected in the collecting flask for about 8 hours. The oil bath was removed, and vacuum line disconnected, the resulting light yellow viscous crude product was poured into a alumina foil tray and solidified as it was cooled to room temperature. The crude product was broken into small pieces and ground into off-white fine powders by a grinder (grinder XF-100, purchased from Shanghai Heqi Glassware Co., Ltd.). The isolated product weighed 490 g and was characterized by TGA and DSC, and the results are listed in Table 2.

### Synthetic Example 2. Preparation of Compound 1a

A mixture of 47.46 g (0.138 moles) of isomalt, 500 g (1.654 moles) of Compound 2a, and 8.21 g (0.059 moles) of potassium carbonate was placed in a 1000 mL three-necked flask equipped with mechanical stirrer, a nitrogen inlet, a condenser and a byproduct collecting flask. The collecting flask was submerged in a dry ice/isopropyl alcohol bath and connected to a vacuum pump. The molar ratio of Compound 2a to isomalt was about 12:1.

The reaction mixture was heated to melt completely at about 170° C. under nitrogen protection with mechanical stirring. After heating at about 170° C. for 1 hour, the reaction temperature was increased to about 190° C. and the nitrogen flow was stopped. The methanol byproduct was removed from the reaction mixture under reduced pressure (about 10⁻¹-10⁻² mbar) and was collected in the collecting flask for about 8 hours. The oil bath was removed, and vacuum line disconnected, the light yellow crude product was cooled and solidified at room temperature. The crude product was broken into small pieces, and dissolved in 500 mL of chloroform in a 1 L beaker. The solution was precipitated in a 5 L beaker that is filled with three liters of ethyl acetate with vigorous stirring. The unreacted Compound 2a was soluble in the solution and separated from the insoluble solids by filtration. The insoluble solids were transferred into a 1000 mL three-neck flask, which was equipped with mechanical stirrer, water condenser and a collecting flask. Remaining solvent was removed from the product by heat at about 190° C. under reduced pressure for 2 hours. After removal the oil bath, the light yellow product was cooled and solidified at room temperature. Finally, the product was broken into small pieces and ground into off-white fine powders. The isolated product weighed 278 g and was characterized by TGA, DSC and ICP, and the results are listed in Table 2.

### Synthetic Example 3. Preparation of Compound 1b

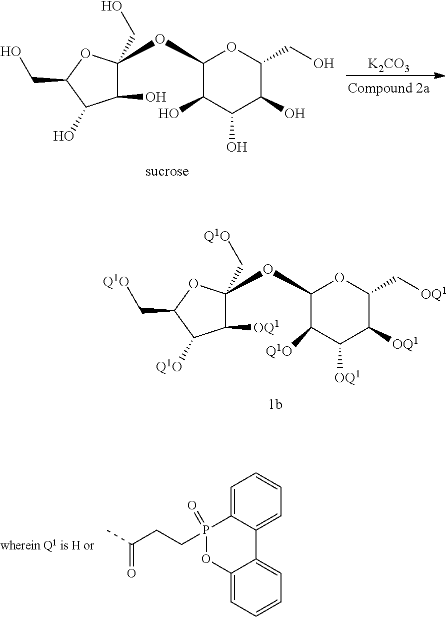

A mixture of 94.36 g (0.276 moles) of sucrose, 500 g (1.654 moles) of Compound 2a, and 8.92 g (0.065 moles) of potassium carbonate was placed in a 1000 mL three-necked flask equipped with mechanical stirrer, a nitrogen inlet, a condenser, and a byproduct collecting flask. The collecting flask was submerged in a dry ice/isopropyl alcohol bath and connected to a vacuum pump. The molar ratio of Compound 2a to sucrose was about 6:1.

The reaction mixture was heated to melt completely at about 135° C. under nitrogen protection with mechanical stirring. After heating at about 135° C. for 2 hours, the reaction temperature was increased to about 160° C. and the nitrogen flow was stopped. The methanol byproduct was removed from the reaction mixture under reduced pressure (about 10⁻¹-10⁻² mbar) and was collected in the collecting flask for about 8 hours. The oil bath was removed, and vacuum line disconnected, the resulting yellow viscous crude product was cooled and solidified as it was cooled to room temperature. The crude product was broken into small pieces and ground into light yellow fine powders. The isolated product weighed 500 g and was characterized by TGA and DSC, and the results are listed in Table 2.

### Synthetic Example 4. Preparation of Compound 1c

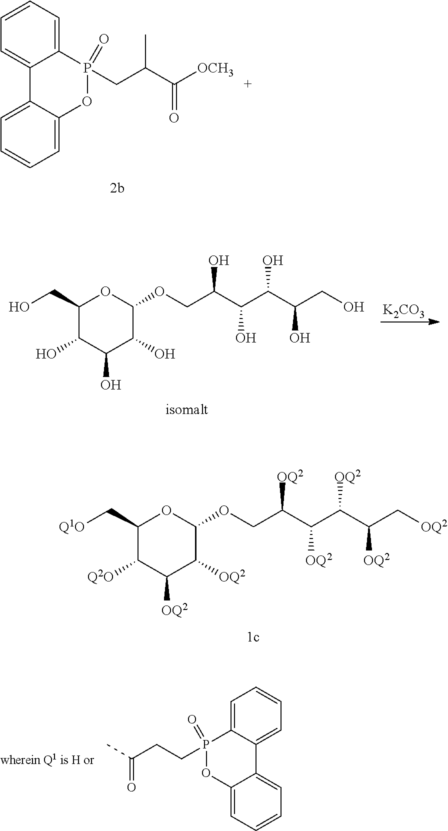

A mixture of 47.51 g (0.138 moles) of isomalt, 500 g (1.592 moles) of Compound 2b, and 8.21 g (0.059 moles) of potassium carbonate was placed in a 1000 mL three-necked flask equipped with mechanical stirrer, a nitrogen inlet, a condenser and a byproduct collecting flask. The collecting flask was submerged in a dry ice/isopropyl alcohol bath and connected to a vacuum pump. The molar ratio of Compound 2a to isomalt was about 12:1.

The reaction mixture was heated to melt completely at about 170° C. under nitrogen protection with mechanical stirring. After heating at about 170° C. for 1 hour, the reaction temperature was increased to about 190° C. and the nitrogen flow was stopped. The methanol byproduct was removed from the reaction mixture under reduced pressure (about 10⁻¹-10⁻² mbar) and was collected in the collecting flask for about 8 hours. The oil bath was removed, and vacuum line disconnected, the light yellow crude product was cooled and solidified at room temperature. The crude product was broken into small pieces, and dissolved in 500 mL of chloroform in a 1 L beaker. The solution was precipitated in a 5 L beaker that is filled with three liters of ethyl acetate with vigorous stirring. The unreacted Compound 2b was soluble in the solution and separated from the insoluble solids by filtration. The insoluble solids were transferred into a 1000 mL three-neck flask, which was equipped with mechanical stirrer, water condenser and a collecting flask. Remaining solvent was removed from the product by heat at about 190° C. under reduced pressure for 2 hours. After removal of the oil bath, the light yellow product was cooled and solidified at room temperature. Finally, the product was broken into small pieces and ground into white fine powders. The isolated product weighed 210 g and was characterized by TGA, DSC and ICP, and the results are listed in Table 2.

### Synthetic Comparative Example 1. (SCE1)

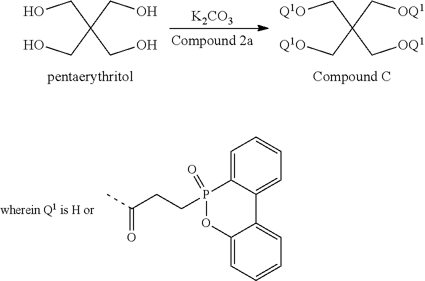

A mixture of 56.30 g (0.414 moles) of pentaerythritol, 500 g (1.654 moles) of Compound 2a, and 8.92 g (0.065 moles) of potassium carbonate was placed in a 1000 mL three-necked flask equipped with mechanical stirrer, a nitrogen inlet, a condenser and a byproduct collecting flask. The collecting flask was submerged in a dry ice/isopropyl alcohol bath and connected to a vacuum pump. The molar ratio of Compound 2a to pentaerythritol was about 4:1.

The reaction mixture was heated to melt completely at about 200° C. under nitrogen protection with mechanical stirring. After heating at about 200° C. for 1 hour, the nitrogen flow was stopped. The methanol byproduct was removed from the reaction mixture under reduced pressure (ca. 10⁻¹-10⁻² mbar) and was collected in the collecting flask for about 8 hours. The oil bath was removed, and vacuum line disconnected, the resulting yellow viscous crude product was poured into a alumina foil tray and solidified as it was cooled to room temperature. The crude product was broken into small pieces and ground into light yellow fine powders. The isolated product weighed 470 g and was characterized by TGA and DSC, and the results are listed in Table 2.

TABLE 2

| | Synthetic Example ID | | | | |
|---|---|---|---|---|---|
| | SE1 | SE2 | SE3 | SE4 | SCE1 |
| Compound ID | compound | compound | compound | compound | compound C |
| | 1a | 1a | 1b | 1c | |
| polyol of Formula 3 | isomalt | isomalt | sucrose | isomalt | isomalt |
| DOPO adduct of | Compound | Compound | Compound | Compound | Compound |
| Formula 2 | 2a | 2a | 2a | 2b | 2a |
| molar ratio of | 7:1 | 12:1 | 6:1 | 12:1 | 4:1 |
| DOPO adduct 2 to | | | | | |
| polyol 3 | | | | | |
| Solvent rinsed | NO | YES | NO | YES | NO |
| T_{d} (° C.) | 355 | 351 | 289 | 345 | 300 |
| Residue at 700° C. | 18 | 18 | 15 | 17.9 | 6.7 |
| (weight %) | | | | | |
| T_{g} (° C.) | 54 | 106 | 34 | 94 | 112 |
| P content (weight | — | 9.4 | — | 8.1 | — |
| %) | | | | | |

Preparation of Flame Retardant Compositions and Test Specimens

According to the amount specified in Tables 3-5, the ingredients of each example and ingredients of each comparative example are processed according to the compounding procedure described below, and tested using general testing methods.

A. Melt Blending

Prior to compounding to keep the moisture content of the pellets less than 20 ppm, the polyester pellets (PTT, PET, and PBT) and the polyamide pellets (PA66, PA610, and PA6) were dried at 80° C. for about 24 hours in a forced air-circulating oven.

The ingredients as specified in Table 3 of E1-E5 and CE1-CE4 having polyesters (PTT, PBT, and/or PET) as the component (a) were fed to a twin screw extruder (Eurolab 16) to obtain the corresponding flame retardant composition as pellets. For polyester pellets containing PTT and PBT, the temperature of the extruder and the die temperature was set to 250° C.; and 270° C. for PET containing pellets. The screw speed was at 200 rpm with a throughput of 2.5 Kg/hour.

The ingredients as specified in Table 4 of E6-E8 and CE5-CE7 having PA610 as the component (a) were fed to a twin screw extruder (Coperion ZSK-26MC) to obtain the corresponding flame retardant composition as pellets. The temperature for the 11 heating block configuration and the die temperature were set to be 240° C. The screw speed was at 300 rpm with a throughput of 20 Kg/hour.

The ingredients as specified in Table 5 of E9-E10 and CE8-CE79 having PA6 as the component (a) were fed to a twin screw extruder (Eurolab 16) to obtain the corresponding flame retardant composition as pellets. The temperature for the 10 heating block configuration and the die temperature were set 250° C. The screw speed was at 200 rpm with a throughput of 2.5 Kg/hour.

The ingredients as specified in Table 5 of E12 and E14 were fed to a mini HAKKI twin screw extruder to obtain the corresponding flame retardant composition as pellets. The melt blending temperature having PA66 as the component (a) were set to 280° C. The screw speed was at 200 rpm with a throughput of 0.5 Kg/hour.

The ingredients as specified in Table 5 of E11, E13 and E10 were fed to a twin screw extruder (Eurolab 16) to obtain the corresponding flame retardant composition as pellets. The melt blending temperature having PA66 as the component (a) were set to 280° C. The screw speed was at 200 rpm with a throughput of 2.5 Kg/hour.

B. Molding

The extruded pellets were dried to a moisture level of less than 40 ppm prior to molding. For flame retardancy testing, the test specimen according to GB/T 2406.2-2009 was molded on a Sumitomo 100 Ton molding machine with a screw diameter of 32 mm and a nozzle diameter of 5 mm. The barrel temperature was set to be 240-280° C. which was the same as their respective melt blending temperature (i.e. for PTT and PBT: 250° C.; for PET: 270° C.; for PA610: 240° C.; for PA6: 250° C.; for PA66: 280° C.), and the molding temperature was 80° C.

The test specimen for mechanical property tests had the basic dumbbell shape, 150 mm long, with the center section 10 mm wide by 4 mm thick by 80 mm long.

The test specimen for flame retardant property tests (LOI) had the rectangle shape with 10 mm wide by 4 mm thick by 80 mm long.

The test specimen for flame retardant property tests (UL94) had the rectangle shape with 13 mm wide by 1.6 mm or 0.8 mm thick by 130 mm long.

Due to small quantity of E12 composite, its test specimens for flame retardant property tests (UL94) were prepared by hot pressing (Gotech Hydraulic Molding Test Presser) and cut to a rectangle of 13 mm wide by 0.8 mm thick by 130 mm long. The general hot pressing procedure was described below.

The extruded pellets were dried to a moisture level of less than 40 ppm prior to pressing. A pile of 30 g of pellets was placed in the center of steel frame (146 mm×146 mm×1 mm) and sandwiched between PTFE sheets. The temperature of the upper pressing plate was set as 285° C., and the temperature of the lower plate was set as 265° C. During the 5 minutes of preheating process, the temperature of the lower plate was gradually increased from 265° C. to 285° C. Gas release in the chamber was implemented twice. When the pressure was applying on the PTFE sheets, 20 MPa pressing pressure was maintained for 2 min, and 40 MPa for 3 min. After release the pressing and peel off the PTFE sheets, a composite sheet was obtained. Finally, the FR PA66 sheet was cut into UL-94 specimens using a steel die cutter.

TABLE 3

| Sample ID | CE1 | E1 | E2 | E3 | CE2 | CE3 | E4 | CE4 | E5 |
|---|---|---|---|---|---|---|---|---|---|
| (a) Thermoplastic | PTT | PTT | PTT | PTT | PTT | PBT | PBT | PET | PET |
| polymer | | | | | | | | | |
| Amount (g) | 2492.5 | 2417.5 | 2417.5 | 2417.5 | 2417.5 | 2492.5 | 2242.5 | 2492.5 | 2242.5 |
| (b) Flame retardant | 0 | SE1 | SE3 | SE4 | ME-P8 | 0 | SE1 | 0 | SE1 |
| source | | | | | | | | | |
| Amount (g) | 0 | 75 | 75 | 75 | 75 | 0 | 250 | 0 | 250 |
| IRGANOX ® 1010, (g) | 7.5 | 7.5 | 7.5 | 7.5 | 7.5 | 7.5 | 7.5 | 7.5 | 7.5 |
| Total weight (g) | 2500 | 2500 | 2500 | 2500 | 2500 | 2500 | 2500 | 2500 | 2500 |
| Flame retardant (b) | 0 | 3 | 3 | 3 | 3 | 0 | 10 | 0 | 10 |
| (weight %) | | | | | | | | | |

TABLE 4

| Sample ID | CE5 | E6 | E7 | E8 | CE6 | CE7 |
|---|---|---|---|---|---|---|
| (a) | PA610 | PA610 | PA610 | PA610 | PA610 | PA610 |
| Thermoplastic | | | | | | |
| polymer | | | | | | |
| Amount (g) | 4000 | 3800 | 3600 | 3400 | 4500 | 4250 |
| (b) Flame | 0 | SE1 | SE1 | SE1 | OP1230 | OP1230 |
| retardant | | | | | | |
| source | | | | | | |
| Amount (g) | 0 | 200 | 400 | 600 | 500 | 750 |
| IRGANOX ® | 20 | 20 | 20 | 20 | 20 | 20 |
| 1098 | | | | | | |
| Total | 4020 | 4020 | 4020 | 4020 | 5020 | 5020 |
| weight (g) | | | | | | |
| Flame | 0 | 5 | 10 | 15 | 10 | 15 |
| retardant (b) | | | | | | |
| (weight %) | | | | | | |

TABLE 5

| Sample ID | CE8 | E9 | E10 | CE9 | CE10 | E11 | E12 | E13 |
|---|---|---|---|---|---|---|---|---|
| (a) Thermoplastic | PA6 | PA6 | PA6 | PA6 | PA66 | PA66 | PA66 | PA66 |
| polymer | | | | | | | | |
| Amount (g) | 2492.5 | 2367.5 | 2367.5 | 2367.5 | 2200 | 2760 | 225 | 2640 |
| (b) Flame retardant | 0 | SE1 | SE3 | SCE1 | 0 | SE2 | SE2 | SE2 |
| source | | | | | | | | |
| Amount (g) | 0 | 125 | 125 | 125 | 0 | 240 | 25 | 360 |
| IRGANOX ® 1098 (g) | 7.5 | 7.5 | 7.5 | 7.5 | 8.8 | 12 | 1 | 12 |
| Total weight (g) | 2500 | 2500 | 2500 | 2500 | 2209 | 3012 | 251 | 3012 |
| Flame retardant (b) | 0 | 5 | 5 | 5 | 0 | 8 | 10 | 12 |
| (weight %) | | | | | | | | |

Flame Retardancy Test

Limiting Oxygen Index (LOI) test was used to evaluate the flame retardancy of the flame retardant compositions. The basic testing and mechanism of LOI test include: placing the test specimen in a transparent cylinder with an upward flowing mixture of nitrogen and oxygen inside, igniting the top of the test specimen to observe the burning, and comparing the continuous burning duration and the burned length with the criteria in relevant standard. A series of tests were done under various oxygen concentrations, and the minimum oxygen concentration needed for burning was recorded.

Limiting Oxygen Index (LOI) test of molded article: in the present invention, LOI of molded article was tested according to the standard of GB/T 2406.2-2009, and the test equipment was from Textile Research Institute, Shandong Province (Model JF-3LSY-605 automatic oxygen index tester), the testing steps are as follows:

Igniting the top of the test specimen of molded article in no more than 30 seconds, if the test specimen cannot be ignited, it indicates that the oxygen concentration is too low. Keep increasing the oxygen concentration until the test specimen is ignited, and observing the burning duration and the burned length. If burning duration is more than 180 seconds, or the burned length is greater than 50 mm, it indicates the oxygen concentration used in the test is the minimum oxygen concentration needed for igniting the test specimen.

Flammability test (UL94): according to the method: “Tests for Flammability of Plastic Materials, UL94”, Underwriter's Laboratory Bulletin 94. Each specimen is mounted with long axis vertical, and is supported such that its lower end is 10 mm above a Bunsen burner tube. A blue 20 mm high flame is applied to the center of the lower edge of the specimen for 10 seconds and removed. If burning ceases within 30 seconds, the flame is reapplied for an additional 10 seconds. If the specimen drips, particles are allowed to fall onto a layer of dry absorbent surgical cotton placed 300 mm below the specimen.

According to this method, based on 5 samples of the test results obtained in 10 flame applications, the rating of flammability of the material is characterized into four levels, including V-0, V-1, V-2, and NVC.

For a V-0 rating, the specimens may not burn with flaming combustion for more than 10 seconds after either application of the test flame, the total flaming combustion time may not exceed 50 seconds for the 10 flame applications for each set of 5 specimens, the specimens may not drip flaming particles that ignite the dry absorbent surgical cotton located 300 mm below the test specimen, and may not have glowing combustion that persists for more than 30 seconds after the second removal of the test flame.

For a V-1 rating, the specimens may not burn with flaming combustion for more than 30 seconds after either application of the test flame, the total flaming combustion time may not exceed 250 seconds for the 10 flame applications for each set of 5 specimens, the specimens may not drip flaming particles that ignite the dry absorbent surgical cotton located 300 mm below the test specimen and may not have glowing combustion that persists for more than 60 seconds after the second removal of the test flame.

For a V-2 rating, the specimens may not burn with flaming combustion for more than 30 seconds after either application of the test flame, the total flaming combustion time may not exceed 250 seconds for the 10 flame applications for each set of 5 specimens, the specimens can drip flaming particles that ignite the dry absorbent surgical cotton located 300 mm below the test specimen and may not have glowing combustion that persists for more than 60 seconds after the second removal of the test flame.

For a NVC (Non-Vertical-Classification) rating, the specimens cannot pass V2 classification in a vertical burning test.

The mechanical properties, such as tensile modulus (0.05%-0.25%) and tensile stress at yield were measured on an Instron 5567 testing system according to ISO 527:1993(E). The reported data is the average of the measured results of 5 test specimens, the standard deviation is generally 1-4%. It is preferable that the specimens have higher values.

The notched Charpy impact strength was measured on a CEAST impact tester using the ISO 179/1eA standard method. The reported data is the average of the measured results of 10 test specimens, the standard deviation is generally 2-10%. The specimens having higher values indicate better impact resistance or toughness.

TABLE 6

| Sample ID | CE1 | E1 | E2 | E3 | CE2 | CE3 | E4 | CE4 | E5 |
|---|---|---|---|---|---|---|---|---|---|
| (a) Thermoplastic polymer | | PTT | PTT | PTT | PTT | PBT | PBT | PET | PET |
| (b) Flame retardant source | 0 | SE1 | SE3 | SE4 | ME-P8 | 0 | SE1 | 0 | SE1 |
| Flame retardant (b), weight % | 0 | 3 | 3 | 3 | 3 | 0 | 10 | 0 | 10 |
| LOI (%) | 23.5 | 28.0 | 27.0 | 30.5 | 24.5 | 23.0 | 24.0 | 23.5 | >26 |
| Tensile modulus (MPa) | 2523 | 2756 | 2951 | 2860 | 2833 | 2760 | 2660 | 3010 | — |
| Tensile stress at break (MPa) | 66 | 64 | 69 | 54 | 67 | 56 | 52 | 70 | — |
| Tensile strain at break (%) | 4.1 | 2.6 | 7.1 | 2.2 | 8.6 | 11 | 3.3 | 3.8 | — |
| N-charpy impact strength (KJ/m²) | 1.5 | 1.4 | 1.4 | 1.5 | 1.5 | 4.5 | 2.5 | 2.5 | — |

From the results of Table 6, the following are evident.

The present compositions E1, E2 and E3, having the same amount of the inventive flame retardant of Formula 1 (i.e. 3 weight % of Compound 1a, Compound 1b and Compound 1c respectively) showed a more significant increase in flame retardancy. In contrast, comparison between the LOI data of CE1 and CE2, it can be seen that when PTT containing 3 weight % of a known polymeric phosphorus-containing flame retardant, ME-P8, the flame retardancy of the CE2 composition increased to 24.5.

Comparison between the mechanical properties data of E1-E3 and CE2 versus that of CE1, it appears that the present flame retardants of Formula 1 may affect the tensile strength and impact strength of the flame retardant composites to an acceptable degree, and which is similar to the effect caused by the known flame retardant, ME-P8.

TABLE 7

| Sample ID | CE5 | E6 | E7 | E8 | CE6 | CE7 |
|---|---|---|---|---|---|---|
| (a) | PA610 | PA610 | PA610 | PA610 | PA610 | PA610 |
| Thermoplastic | | | | | | |
| polymer | | | | | | |
| (b) Flame | 0 | SE1 | SE1 | SE1 | OP1230 | OP1230 |
| retardant | | | | | | |
| source | | | | | | |
| Flame | 0 | 5 | 10 | 15 | 10 | 15 |
| retardant (b) | | | | | | |
| (weight %) | | | | | | |
| UL-94 | NVC | NVC | V-2 | V-0 | V-2 | V-2 |
| (1.6 mm thick) | | | | | | |
| LOI (%) | 24.0 | 25.0 | 27.0 | 28.5 | — | — |
| Tensile | 2650 | 2660 | 2590 | 2530 | 2765 | 2940 |
| modulus (MPa) | | | | | | |
| Tensile stress | 43 | 56 | 69 | 71 | 54 | 53 |
| at break | | | | | | |
| (MPa) | | | | | | |
| Tensile strain | 20 | 12 | 5.0 | 3.6 | 17 | 10 |
| at break (%) | | | | | | |
| N-charpy | 4.3 | 2.8 | 2.7 | 1.4 | 2.5 | 2.5 |
| impact strength | | | | | | |
| (KJ/m²) | | | | | | |

From the results Table 7, the followings are evident.

Comparison between the LOI data of E6-E8 and CE5, by increasing the amount of the present flame retardant (i.e. Compound 1a) from 5 weight % to 15 weight %, the flame retardancy of the respective compositions of E6, E7 and E8 increased from 25.0 to 28.5. Furthermore, the flame retardancy of these compositions are also confirmed by the UL-94 test results. By adding 15 weight % of Compound 1a to the PA610, the resulting composition of E8 showed a V-0 rating, which is the highest rank of the UL-94 test.

In contrast, when PA610 containing 5 weight % or 10 weight % of a known phosphinate salt type flame retardant, i.e. OP1230, the flame retardancy of CE6 and CE7 versus CE5 only increased from NVC to V-2.

TABLE 8

| | Sample ID | | | |
|---|---|---|---|---|
| | CE8 | E9 | E10 | CE9 |
| (a) Thermoplastic polymer | PA6 | PA6 | PA6 | PA6 |
| (b) Flame retardant source | 0 | SE1 | SE3 | SCE1 |
| Flame retardant (b) | 0 | 5 | 5 | 5 |
| (weight %) | | | | |
| LOI (%) | 23.0 | 24.0 | 24.5 | 24.0 |
| Tensile modulus (MPa) | 2788 | 3188 | 3500 | 3560 |
| Tensile stress at break (MPa) | 70 | 74 | 73 | 76 |
| Tensile strain at break (%) | 5.2 | 3.4 | 3.2 | 3.1 |
| N-charpy impact strength (KJ/m²) | 4.2 | 2.6 | 2.6 | 2.6 |

From the results Table 8, the followings are evident.

Comparison between the LOT data of E9-E10 and CE8, by adding 5 weight % of the present flame retardant (i.e. compound 1a and compound 1b, respectively) to PA6, the flame retardancy of the compositions of E9 and E10 increased to 24.0 and 24.5, respectively. In contrast, when PA6 containing 5 weight % of the compound C (i.e. CE9), the flame retardancy of CE9 is comparable to that of the E9, but not as good as that of E10.

TABLE 9

Sample ID

| | CE10 | E11 | E12 | E13 |
|---|---|---|---|---|
| (a) Thermoplastic polymer | PA66 | PA66 | PA66 | PA66 |
| (b) Flame retardant source | 0 | SE2 | SE2 | SE2 |
| Flame retardant (b) | 0 | 8 | 10 | 12 |
| (weight %) | | | | |
| UL-94 (0.8 mm thick) | V-2 | V-0 | V-0 | V-0 |

From the results of Table 9, the following are evident.

Comparison between the UL-94 data of E11-E13 versus CE10, it can be seen that when PA66 containing 8-12 weight % of the present flame retardant (i.e. Compound of 1a), the present compositions (i.e. E11-E13) showed more significant improvement in flame retardancy to obtain a V-0 rating of the UL-94 test.

While the invention has been illustrated and described in typical embodiments, it is not intended to be limited to the details shown, since various modifications and substitutions are possible without departing from the spirit of the present invention. As such, modifications and equivalents of the invention herein disclosed may occur to persons skilled in the art using no more than routine experimentation, and all such modifications and equivalents are believed to be within the spirit and scope of the invention as defined by the following claims.

1. A flame retardant comprising a compound of Formula 1: 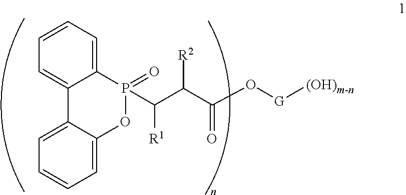
wherein
G-(OH)ₘ is a disaccharide or a C₁₂ sugar alcohol, which has at least one glucose or one fructose unit per molecule;
R¹ is H or CH₃;
R² is H or CH₃;
m is an integer ranging from 6 to 9; and
n is an integer ranging from 2 to 9.

2. The flame retardant of claim 1, wherein the polyol is selected from the group consisting of cellobiose, lactose, lactulose, maltose, sucrose, trehalose, gentiobiose, gentiobiulose, isomaltose, kojibiose, laminaribiose, maltulose, mannobiose, melibiose, melibiulose, nigerose, palatinose, rutinose, rutinulose, sophorose, turanose, xylobiose, isomalt, lactitol, and maltitol.

3. The flame retardant of claim 2, wherein the polyol is selected from the group consisting of sucrose and isomalt and n is an integer ranging from 4 to 8.

4. A method of preparing a compound of Formula 1, comprising: 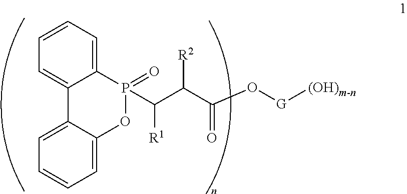
wherein
G-(OH)ₘ is a disaccharide or a C₁₂ sugar alcohol, which has at least one glucose or one fructose unit per molecule;
R¹ is H or CH₃;
R² is H or CH₃;
m is an integer ranging from 6 to 9; and
n is an integer ranging from 2 to 9;
i) forming a reaction mixture comprising an organophosphorus compound of Formula 2, a polyol of Formula 3, and a base, 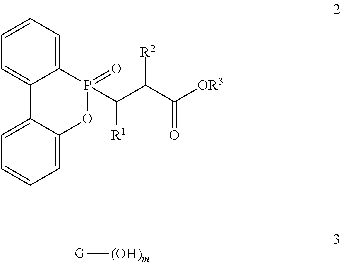
wherein
R¹ is H or CH₃;
R² is H or CH₃; and
R³ is H or C₁-C₄ alkyl;
the polyol is a disaccharide or a C₁₂ sugar alcohol, which has at least one glucose or one fructose unit per molecule; and
m is an integer ranging from 6 to 9;
ii) heating the reaction mixture at 125-230° C. and a pressure of 0.01-100 mbar for 8-24 hours; and
iii) isolating a mixture of the compound of Formula 1.

5. The method of claim 4, wherein R3 is CH3 or C2H5, the base is sodium carbonate, potassium carbonate, calcium carbonate, barium carbonate, sodium bicarbonate, potassium bicarbonate, or mixtures thereof, and the molar ratio of base to the polyol of Formula 3 is from about 0.01:1 to about 0.1:1.

6. A thermoplastic composition having improved flame retardancy, comprising:
a) 80 to 99.9% by weight of a thermoplastic polymer, and
b) 0.1 to 20% by weight of the flame retardant of claim 1,
wherein the % is based on the total weight of the thermoplastic composition.

7. The thermoplastic composition of claim 6, wherein the thermoplastic polymer is selected from the group consisting of polyesters, polyamides, polyurethanes, polyolefins, and blends thereof.

8. The thermoplastic composition of claim 7, wherein the thermoplastic polymer is selected from polyesters, polyamides, and blends thereof.

9. The thermoplastic composition of claim 8 further comprising at least one additive selected from the group consisting of antioxidants, thermal stabilizers, ultraviolet light stabilizers, colorants including dyes and pigments, lubricants, hydrolysis resistants, anti-dripping agents, fillers, and demolding agents.

10. An article, comprising or produced from the thermoplastic composition of claim 6.

11. A method for improving flame retardancy of a thermoplastic polymer comprising: incorporating a flame retardant of claim 1 into the thermoplastic polymer.

12. The method of claim 11, wherein the flame retardant is incorporated into the thermoplastic polymer by a melt blending process.

13. The method of claim 12, wherein 0.1-20% by weight, or 5-15% by weight, of the flame retardant is incorporated into the thermoplastic polymer, based on the combined weight of the flame retardant and the thermoplastic polymer.
